(19) United States
(12) Patent Application Publication
(10) Pub. No.: US 2015/0018666 A1
(43) Pub. Date: Jan. 15, 2015

(54) Method and Apparatus for Registering Image Data Between Different Types of Image Data to Guide a Medical Procedure

(71) Applicants: Anant Madabhushi, Shaker Heights, OH (US); Rachel Sparks, London (UK)

(72) Inventors: Anant Madabhushi, Shaker Heights, OH (US); Rachel Sparks, London (UK)

(21) Appl. No.: 14/330,582

(22) Filed: Jul. 14, 2014

Related U.S. Application Data
(60) Provisional application No. 61/845,678, filed on Jul. 12, 2013.

(51) Int. Cl. A61B 8/08; A61B 10/02; G01R 33/28; A61B 18/20; G01R 33/48; A61B 8/00; A61B 10/04

(52) U.S. Cl. CPC A61B 8/5261; A61B 8/463; A61B 8/0841; A61B 10/0233; A61B 10/04; A61B 18/20; G01R 33/4814; G01R 33/285; A61B 2010/045; A61B 2018/2005; A61B 5/055
USPC 600/411

(57) ABSTRACT

A method and apparatus are provided for registering image data between two different types of image data. The image data is obtained during two separate scanning processes. The first process is a high-resolution imaging process, such as a CT scan or an MRI. The second process is a lower resolution process, such as ultrasound. The image data is registered in real time so that the combined image data can be displayed during the second process to guide a medical professional during a procedure.

### PRIORITY CLAIM

This application claims priority to U.S. Provisional App. No. 61/845,678 filed on Jul. 12, 2013. The entire disclosure of the foregoing application is hereby incorporated herein by reference.

### FIELD OF THE INVENTION

The present invention relates to the field of image processing. In particular, this application relates to the field of registering portions of one image with a portion of one or more separate images. The images are registered in real time to guide a medical procedure, such as a biopsy or treatment.

### BACKGROUND

In fields, such as the medical industry, it is useful to correlate image data from one image with image data from a separate modality. For instance, in the medical field, there are numerous types of images that may be used during diagnostics or treatment for a patient, including MRI, CT scans, ultrasound, x-rays and others. These various imaging modalities each have advantages and disadvantages. Furthermore, medical professionals may have different reasons for utilizing multiple imaging modalities for the same anatomical region of the patient. However, manually correlating the image data from the two types of image modalities can be difficult or impossible in many instances.

For instance, Transrectal Ultrasound (TRUS)-guided needle biopsy is the current gold standard for prostate cancer diagnosis. However, up to 40% of prostate cancer lesions appear isoechoic on TRUS, meaning that the cancer lesion appears similar to normal or healthy tissue. Hence TRUS-guided biopsy has a high false negative rate for prostate cancer diagnosis. Magnetic Resonance Imaging (MRI) is better able to distinguish prostate cancer lesions from benign prostatic tissue, but MRI-guided biopsy requires specialized equipment and training, and longer procedure times. MRI-TRUS fusion, whereby MRI is acquired pre-operatively and then aligned to TRUS, allows for the advantages of both modalities to be leveraged during the biopsy procedure. Combining MRI and TRUS to guide biopsy offers the potential to substantially increase the yield of cancer positive biopsies.

### SUMMARY OF THE INVENTION

The present invention provides a system for registering imaging data from one modality with image data from a second modality. For instance, the present system provides a method for automatically co-register in vivo MRI and ultrasound images or mechanical images to improve diagnosis and/or treatment by a medical professional.

According to one aspect, the present invention provides a method for registering image data between two images. The method includes the step of scanning a patient using a first imaging modality to obtain a first set of image data for a portion of the patient and scanning a patient using a second imaging modality to obtain a second set of image data for the portion of the patient. A portion of the first set of image data corresponding to a portion of interest is identified and then the second set of data is analyzed relative to the identified portion to identify a portion of the second image data corresponding to the portion of interest. The corresponding portions of the first and second image data can then be simultaneously displayed.

According to another aspect, the present invention provide a method for registering image data of a patient from two imaging devices to guide a surgical procedure. The first set of image data of the patient is provided by a first imaging device using a first imaging modality. The method comprises the steps of scanning a patient, processing the image data and then displaying the image data. In particular, the step of scanning comprises scanning a patient using a second imaging device to obtain a second set of image data for a portion of the patient. The second imaging device uses a second imaging modality that is different than the first imaging modality. The step of processing the image data comprises automatically processing the second set of image data to correlate the second set of image data with the first image data modality to align image data of a target tissue of the patient from the second imaging modality with image data of the portion of tissue from the first imaging modality. The step of displaying the image data comprises displaying the aligned image data from the first and second image data.

According to another aspect, the present invention provides a surgical apparatus. The apparatus includes a scanning element, a surgical element, such as a cutting element, a scanning element, an image processor and a display. The scanning element is configured to use a first imaging modality to scan a patient to obtain a first set of image data from a patient. The image processor is configured to process the first set of image data and correlate the first set of image data with a second set of image data from the patient obtained using a second imaging modality. The image processor processes the first set of image data to align image data of a target tissue of the patient from the scanning element with image data of the portion of tissue from the second set of image data.

### DESCRIPTION OF THE DRAWINGS

The foregoing summary and the following detailed description of the preferred embodiments of the present invention will be best understood when read in conjunction with the appended drawings, in which:

FIG. 1 is a diagrammatic view of a surgical apparatus;

FIG. 2 is a view of an MRI image with a portion of interest identified by a green boundary;

FIG. 3 is a sequence of images illustrating a prostate location on a transrectal ultrasound;

FIG. 4A is an illustration of the registration of an MRI prostate segmentation to the estimated prostate location on TRUS;

FIG. 4B illustrates a mosaic formed of MRI and TRUS image segments showing the correlation between MRI data and the portions of the identified area of the TRUS images.

FIG. 5(A) illustrates MRI image data from a first patient;

FIG. 5(b) illustrates TRUS image data from the first patient;

FIG. 5(C) illustrates MRI image data from a second patient;

FIG. 5(D) illustrates TRUS image data from the first patient;

FIG. 6 is a flowchart illustrating modules of a process for registering MRI image data with TRUS image data

FIG. 7 is a flowchart for the semi-automated prostate segmentation scheme;

FIG. 8(A) is an ultrasound image;

FIG. 8(B) is the ultrasound image of FIG. 8(A) illustrating the corresponding median feature;

FIG. 8(C) is the probability model of the ultrasound image of FIG. 8(A) in which blue corresponds to those pixels least likely to belong to the prostate and red corresponds to those pixels most likely to belong to the prostate;

FIG. 8(D) is the combine display of portions of the ultrasound image of FIG. 8(A) with corresponding portions of MRI images;

FIG. 8(E) is the ultrasound image with attenuation correction;

FIG. 8(F) is the ultrasound image of FIG. 8(E) illustrating the corresponding median feature;

FIG. 8(G) is the probability model of the ultrasound image of FIG. 8(E) in which blue corresponds to those pixels least likely to belong to the prostate and red corresponds to those pixels most likely to belong to the prostate;

FIG. 8(H) is the combine display of portions of the ultrasound image of FIG. 8(E) with corresponding portions of MRI images;

FIG. 9(A) is an ultrasound image;

FIG. 9(B) is the ultrasound image of FIG. 9(A) processed using a median texture feature;

FIG. 9(C) is the ultrasound image of FIG. 9(A) processed using a range texture feature;

FIG. 9(D) is the ultrasound image of FIG. 9(A) processed using a Rayleigh texture feature;

FIG. 9(E) is the ultrasound image of FIG. 9(A) processed using a Gabor wavelet texture feature;

FIG. 10(A) is a graphical illustration of the regularization constraint R(T);

FIG. 10(B) is a graphical illustration of the regularization constraint R(T) in which R(T) would have a high value because p¹ is far from E[p¹];

FIG. 10(C) is a graphical illustration of the regularization constraint R(T) in which R(T) would have a lower value because p¹ is near E[p¹];

FIG. 10(D) is a graphical illustration of the regularization constraint R(T) in which R(T) would have a lower value because p¹ is near E[p¹], wherein the deformation not local to p¹ is not taken into account when considering E[p¹];

FIG. 11 is a graphical illustration of the RMSE for 5 texture features with and without attenuation correction;

FIG. 12(A) is a graphical illustration of the RMSE for D1 as a function of the Rayleigh feature;

FIG. 12(B) is a graphical illustration of the RMSE for D1 as a function of the variance feature;

FIG. 13 (A) is a graphical illustration of the RMSE for Te and Ta evaluated over 5 representative texture features for D₁;

FIG. 13 (B) is a graphical illustration of the RMSE for Te and Ta evaluated over 5 representative texture features for D₂;

FIG. 14(A) is an MRI image;

FIG. 14(B) is an ultrasound image corresponding to the portion of the patient illustrated in FIG. 14(A)

FIG. 14(C) is an overlay registering portions of the MRI image of FIG. 14(A) and the ultrasound image of FIG. 14(B) in which the images were registered for D₁ for Tₑ

FIG. 15(A) is an MRI image;

FIG. 15(B) is an ultrasound image corresponding to the portion of the patient illustrated in FIG. 15(A)

FIG. 15(C) is an overlay registering portions of the MRI image of FIG. 15(A) and the ultrasound image of FIG. 15(B) in which the images were registered for D₂ for Tₑ;

FIG. 16 is a graphical illustration of RMSE for Te evaluated over five representative texture features: Gabor wavelet, intensity, median, Rayleigh, and variance, for two different radiologists for D₂;

FIG. 17(A) is a surface rendering of a prostate;

FIG. 17(B) is an ultrasound image displaying a region of misalignment distal to the ultrasound probe;

FIG. 17(C) is an ultrasound image displaying a region of misalignment near the ultrasound probe;

FIG. 18(A) is a graphical illustration of RMSE as a function of prostate segmentation scheme evaluated using intensity texture feature for D₁;

FIG. 18(B) is a graphical illustration of RMSE as a function of prostate segmentation scheme evaluated using variance texture feature for D₁;

FIG. 18(C) is a graphical illustration of RMSE as a function of prostate segmentation scheme evaluated using intensity texture feature for D₂; and

FIG. 18(D) is a graphical illustration of RMSE as a function of prostate segmentation scheme evaluated using Rayleigh texture feature for D₂.

### DETAILED DESCRIPTION OF THE INVENTION

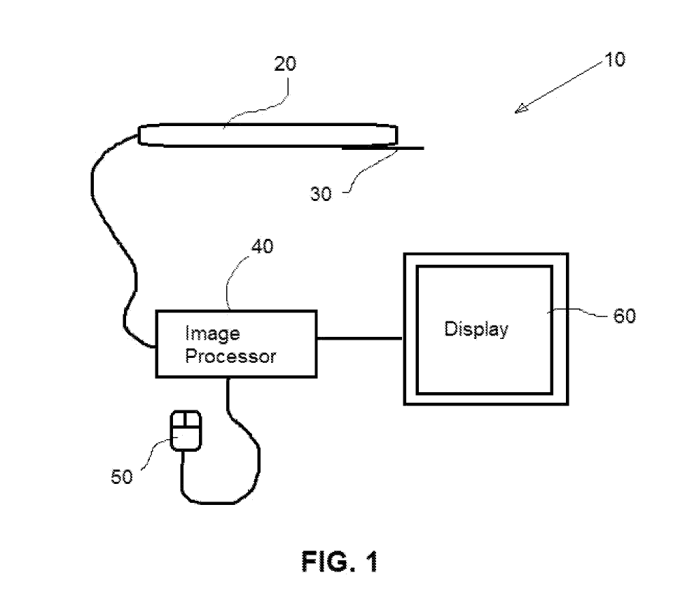

Referring now to the figures in general and to FIG. 1 in particular, a system for guiding a biopsy needle is designated generally 10. The system includes an imaging device 20, such as an ultrasound probe. A surgical instrument 30 is also provided for obtaining tissue samples. For instance, the surgical instrument may be a biopsy needle or a laser probe for treatment. An image processor 40 processes image data from the imaging device 20 and provides images on a display 60 to guide the positioning of the biopsy needle 30 or laser probe. The system 10 also includes one or more input mechanisms 50 so that the operator can input information to the system. For instance, the input mechanism can be any of a variety of input devices, including, but not limited to: a mouse, a stylus, a keyboard and a touch screen.

During use, the operator controls the positioning of the imaging device 20 to obtain image data of areas of interest of a patient. The image data is used to identify patient tissue of interest so that the tissue can be biopsied. The image data is displayed in real time so that the medical professional can use the images to identify tissue to be biopsied or treated. The displayed images are used to navigate the biopsy needle into position so that the biopsy needle can take a sample of the tissue or for a laser probe to selectively ablate specific tissue regions.

In the following discussion, the imaging device 20 is an ultrasound probe, however, it should be understood that other imaging devices may be used to obtain real time imaging data to guide the positioning of the surgical tool.

Although a variety of image processors may be used, in the present instance, the image processor 40 comprises a microprocessor, such as a personal computer. The image processor is configured to obtain image data from the imaging device 20, process the image data and display the data in real time on the display. As discussed below, the image processor 40 is also operable to correlate the real time image data from the imaging device with image data that was previously obtained for the patient for the same area of interest of the patient. By correlating the real time image data with the previously obtained image data, the image processor can display images that more clearly identify certain tissue, such as lesions.

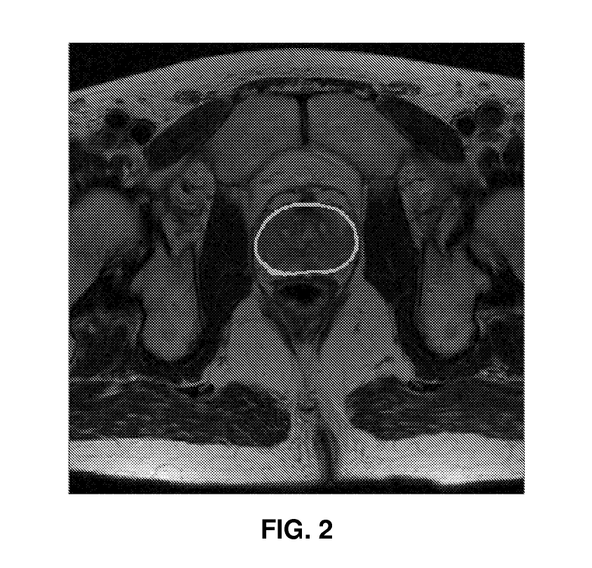

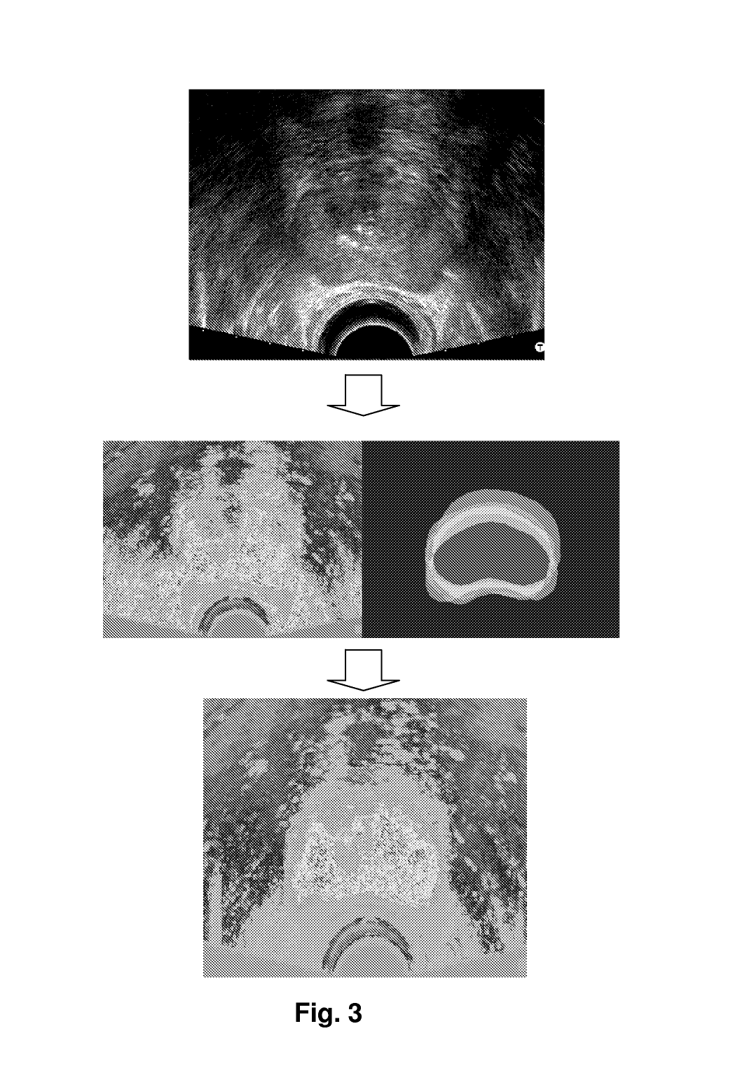

Referring now to FIGS. 2-4B, a method for automatically co-registering two image modalities is provided. In FIG. 2, an in vivo MRI of a patient's prostate is presented. In FIG. 3, an in vivo TRUS image of the patient's prostate is presented. A TRUS guided biopsy is the current standard for prostate cancer (PCa) diagnosis. However, TRUS imaging can be more difficult for medical professionals to distinguish between PCa and benign tissue. In contrast, an MRI has a higher predictive positive value in detecting PCa. Nonetheless, MRI images are more expensive and time-consuming to obtain than TRUS images because TRUS imagery can be obtained in a doctor's office while MRI image acquisition necessitates a separate facility. Accordingly, accurate spatial alignment of MRI to TRUS may aid in localization of PCa and may increase PCa detection.

To register two images, a first image is obtained using the first imaging modality. For example, a patient may be scanned to obtain an MRI of a region of interest. The image data for the MRI is then analyzed to identify a region of interest for a patient. One example would be to have an operator, such as a trained medical professional examine the MRI image and manually select a portion of the MRI image relating to a region of interest. For instance, the operator may circumscribe an area of the MRI image relating to an organ or other area of interest of the patient, such as a region suspicious for disease. The operator may circumscribe the region using any of a variety of input mechanisms, such as a stylus, mouse, or touch screen. Additionally, the operator may use the imaging processor 40 to select the subsets of data from the MRI images or the operator may identify the relevant subsets of data separately and then the image data may be imported into the system 10 to be correlated with the real time image data during the TRUS procedure as described further below.

By identifying the region of interest in the MRI data, the operator identifies the portion of the image data from the MRI relating to the region of interest of the patient. Information from this subset of the MRI image data is then registered with the image data for a second modality, such as ultrasound image data.

The image data for the second modality is then analyzed to evaluate which portion of the second image corresponds with the select portion of the first image data (e.g. the select MRI image data). A variety of systems can be used to analyze the image data. For instance, each pixel of the second image data can be evaluated by creating a window around the pixel and evaluating one or more characteristics of the pixel as well as the relation of the pixel relative to the other pixels in the window (i.e. neighboring or adjacent pixels). Additionally, for each pixel a variety of features can be evaluated. Exemplary features include first order statistics (such as mean, median, standard deviation, range, x-gradient, and y-gradient relative), second order statistics (such as contrast energy, contrast inverse moment, contrast average, contrast variance, contrast entropy, intensity average, intensity variance, intensity entropy, entropy, energy, correlation, information measure of correlation) and information measure of correlation 2), edge statistics, steerable edge statistics and statistics that are particular to the particular image type, such as ultrasound or mechanical specific statistics.

By analyzing various features of the ultrasound or mechanical imaging data, a portion of the image data from the second modality is identified as correlating with the portion of the MRI image that was identified as a region of interest. In this way, the operator can see in real-time or near real-time, which area of an image relates to a region of interest in the patient.

The process for registering the images comprises first obtaining a first set of image data for a portion of the patient. In the present instance, the first modality is an in vivo MRI image scan. The image data is then analyzed to identify a subset of the MRI image data that relates to a region of interest. In the present instance, the area of interest is the prostate and a medical professional manually identifies the relevant subset of data by marking various landmarks to circumscribe the region of interest, as shown in FIG. 2. A second set of image data is then obtained for the patient. In the present instance, the second image data is image data from a TRUS scan. As shown in FIG. 3, the TRUS data is analyzed to estimate the location of the area of interest, which in the present instance is the prostate. The TRUS data is analyzed first by analyzing image intensity, with the results being shown in the left image of the middle row of images in FIG. 3. The TRUS data is also analyzed according to spatial location, with the results being shown in the right image of the middle row of images in FIG. 3. In the spatial location analysis, the red color corresponds to likely prostate locations and the blue color corresponds to unlikely prostate locations.

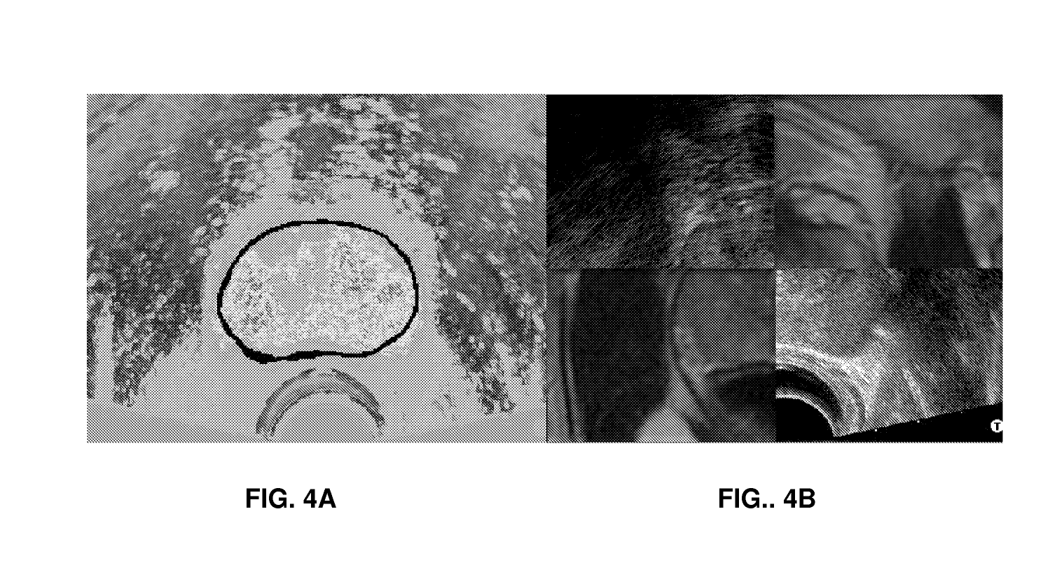

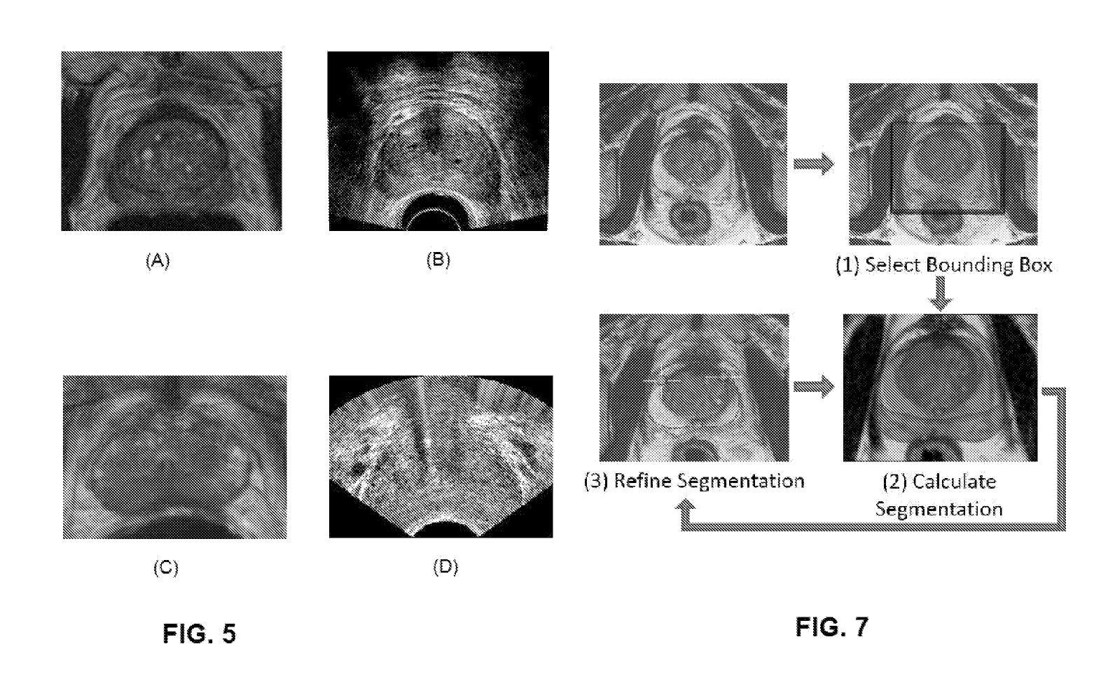

The two estimates based on the analysis of the TRUS data, as discussed above, are combined to obtain a single estimate of the prostate location on the TRUS image, as illustrated in the bottom row of FIG. 3. FIG. 4A illustrates registration of the MRI prostate segmentation to the estimated location on the TRUS image, with the contour of the prostate shown in black. FIG. 4B illustrates a mosaic formed of MRI and TRUS image segments showing the correlation between the MRI data and the portions of the identified area of the TRUS images. The MRI image segments are shown in the upper right and lower left quadrants of the mosaic, while the TRUS image segments are shown in the upper left and lower right quadrants of the mosaic. As can be seen, the mosaic shows a good alignment between the selected MRI image data and the identified portion of the TRUS image data.

The details of a particular methodology for registering a set of image data from a first image with a subset of image data from a second image will now be described. According to the present system an image C=(C, f) where C is a set of coordinates c ε C, and f(c) is a vector of feature values of C at location c. For example one image may be an MRI image C_{MRI} and one image may be a TRUS image C_{TRUS}. Each image contains a prostate denoted by S_{MRI} or S_{TRUS} on MRI and TRUS respectively.

The goal of registration is to find a set of transformation parameters denoted T. T is defined such that the MRI image can be mapped to the TRUS image by T(C_{MRI})=C TRUS. T is found via minimizing,

T=argmax[ψ(T(C_{MRI}),C_{TRUS})] (1)

where ψ is an image similarity measure. Traditional functions such as ψ rely on correspondence between feature values, such as mean square error, or statistical correlations, such as mutual information. These are inappropriate for registration between MRI and TRUS images. The differences in image acquisition result in f_{MRI}(c) and f_{TRUS}(c) not having intensity correspondence or correlation.

Assuming that T will result in an accurate overlap of T(S_{MRI}) and S_{TRUS}.

Equation 1 can be rewritten as,

T=argmax[ψ(T(S_{MRI}),S_{TRUS})] (2)

C_{MRI} is acquired pre-operatively, therefore S_{MRI}, is accurately delineated either via an automated scheme or by a medical professional identifying the portion of the MRI image data corresponding to the portion of the MRI of interest (i.e. the prostate in the example). However, C_{TRUS} is acquired inter-operatively. Ensuring accurate estimation of S_{TRUS} on C_{TRUS} is time consuming. Since S_{TRUS} is unknown it can be estimated.

To relate this back to the registration, T should be estimated so that T(S_{MRI})=Ŝ_{TRUS}. From this ψ can be formulated as,

ψ=P(T(S_{MRI})|C_{TRUS}). (3)

Equation 3 can be rewritten using Bayes rules as,

$$ψ = P ( 𝒯 ( {𝒮}_{MRI} ) \vert {𝒞}_{TRUS} ) = \frac{P ( 𝒯 ( {𝒮}_{MRI} ) ) P ( {𝒞}_{TRUS} \vert 𝒯 ( {𝒮}_{MRI} ) )}{P ( {𝒞}_{TRUS} )} \qquad ( 4 )$$

As the denominator (P(C_{TRUS})) of ψ is not dependent on T it can be ignored. Additionally, assuming P(T(S_{MRI})) is equal across all instances. Hence equation 4 becomes a maximum likelihood estimation (MLE). In other words,

ψ=P(C_{TRUS}|T(S_{MRI})) (5)

Considering each vector f(c) conditionally independent and assuming T(S_{MRI}) divides the image into two discrete classes {Λ₁,Λ₂}. Equation 5 can be estimated as

P(C_{TRUS}|T(S_{MRI}))=Πᵢ₌₁²Π_{cεC}_{ᵢ}P(f(c)|T(S_{MRI})=Λᵢ) (6)

Each conditional probability is assumed to be defined by some distribution Dᵢ. To perform a maximum a posterior estimate (MAP) equation 6 is used to express the log of the a posteriori probability of the form,

P(C_{TRUS}|T(S_{MRI}))=Σᵢ₌₁²Σ_{cεC}_{ᵢ}P(F(c)|T(S_{MRI})=Λᵢ) (7)

To implement Equation 7 the following procedure is used

(1) Given an estimation of T^{k}(S_{MRI}) find approximations of D₁^{k} and D₂^{k}.

(2) Approximate T^{k+1}(S_{MRI}) using Equation 7, D₁^{k} and D₁^{k}.

### Example 1

A T2 weighted MRI was acquired using a Siemens 1.5 T scanner and a pelvic phase array coil for seven patients. Three dimensional TRUS imagery was acquired using a bi-planar side-firing transrectal probe. A radiologist segmented each prostate and selected 3-5 landmarks on MRI and TRUS images. Registration was performed by segmenting the prostate in the MRI manually. The prostate location was then estimated for the TRUS image. The identified MRI portion is then registered with the estimated TRUS portion. The registration can be evaluated using any of a number of measurements or analytics. In the present instance, registration was evaluated using the sum of square differences of landmarks and prostate overlap. In the present instance, the measured overlap was 5.85±1.08 mm and an overlap of 73.6±4.7%

Automated Image Registration

In addition to the process described above, the process of registering the MRI and TRUS data can be semi-automated or fully automated. An automated methodology for registering the MRI and TRUS data described further below is referred to as Multi-Attribute Probabilistic Prostate Elastic Registration (MAPPER). The MAPPER process is a software based process controlled by the image processor 40.

The MAPPER process automatically registers TRUS image data with the MRI image data during the biopsy procedure without manual intervention. The MAPPER process include estimation of the location of the prostate on TRUS and an image registration metric that aligns a binary mask of the prostate to a probabilistic map of its location.

As discussed further below, the MAPPER process allows for estimation of the location of the prostate on TRUS by creating a probabilistic map of the prostate location by combining texture and spatial priors pertaining to gland appearance. This approach calculates a probabilistic map of the prostate location on TRUS image in order to facilitate registration.

The spatial prior, which is calculated from a set of training images, describes the likelihood of a pixel belonging to the prostate according to its spatial location relative to the TRUS probe. The texture prior, calculated as a Gaussian model from a set of texture features, describes the likelihood of a pixel belonging to the prostate according to its local intensity and texture properties. Among other features, the MAPPER process include:

(1) consideration of local intensity and texture properties;

(2) each pixel has a continuous probability value contained in the range of 0 to 1; and

(3) the texture and spatial priors are assumed to be independent.

The choice of texture features used to calculate the texture prior affects the accuracy of the MAPPER process. Texture features which distinguish between prostate and background pixels will result in a more accurate registration compared to texture feature which are unable to distinguish between prostate and background pixels. A variety of texture features can be used, including, but not limited to: first-order texture features (mean, median, range, variance), edge detecting texture features (Gabor wavelet), and ultrasound specific features (Rayleigh, Nakagami m-parameter).

The inclusion of texture-based probability makes the MAPPER registration metric sensitive to TRUS image appearance. Hence, it is desirable for the TRUS imagery to have a consistent appearance, in terms of pixel intensity and texture characteristics. However, ultrasound imagery may have attenuation artifacts, where pixels closer to the ultrasound probe appear brighter than pixels far away. Attenuation is caused by signal loss as the ultrasound waves propagate through tissue. Because the TRUS probe is circular, variations in image intensity will be along radial lines from the probe. To account for changes in attenuation, correction methods may be applied to facilitate and improve image registration.

The MAPPER process also provides a registration metric to align a binary shape onto a probabilistic model for registration of the MRI segmentation to the probabilistic map of the prostate location on TRUS. The similarity metric is calculated by combining the probability of individual pixels belonging inside and outside the prostate to maximize the likelihood of accurate alignment of the prostate segmentation on MRI to the probabilistic map of prostate location on the TRUS image data. This similarity metric should return a high value for transformations where regions inside the MRI prostate segmentation align with pixels that have a high probability of being prostate. Conversely, the similarity metric should yield a low value for transformations where regions inside the MRI prostate segmentation align with pixels that have a low probability of being prostate.

Methodology

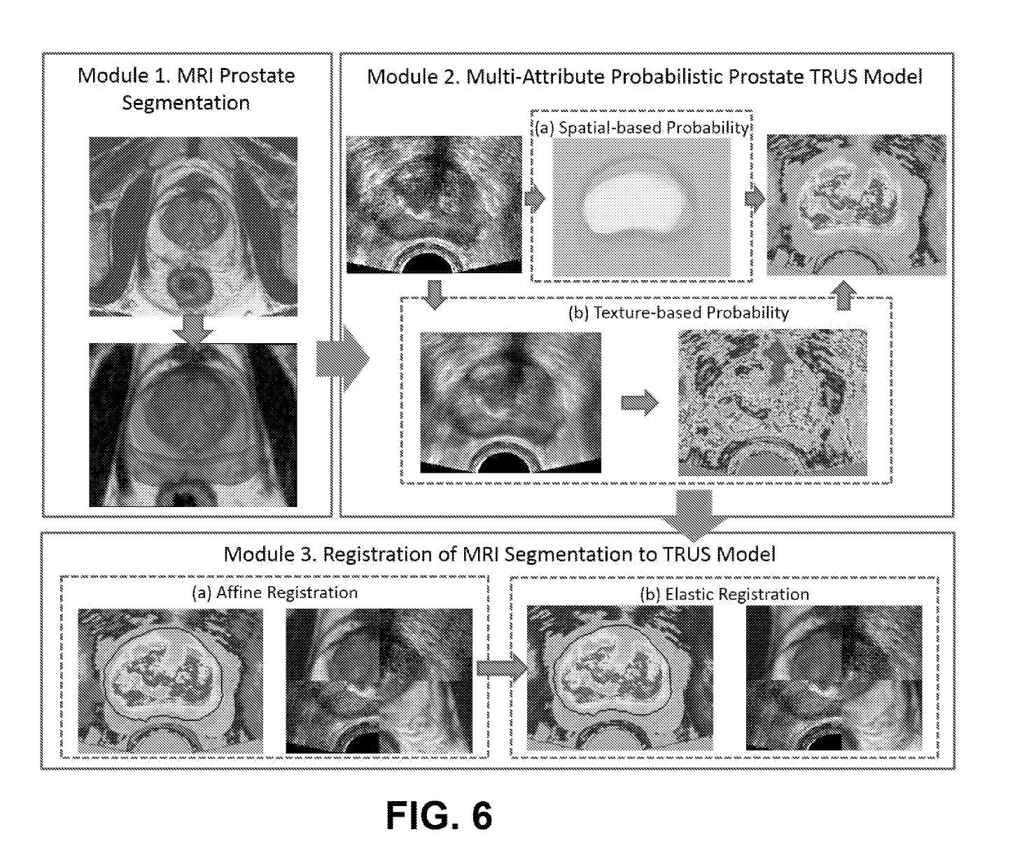

A 3D MRI volume c_{M}=C_{M}, f_{M} is defined by a set of 3D Cartesian coordinates and the image intensity function f_{M}(c): c ε C_{M}. The 3D prostate segmentation result is represented by M_{M}=C_{M}g_{M} such that g_{M(c)}=i for a pixel i belonging to class i, where i=1 represents the prostate and i=0 represents the background. A 3D TRUS volume C_{T}=C_{T},f_{T} is defined in a similar way as C_{M}. From C_{T} a probabilistic map C_{P,i}=C_{T},P_{i(c)} is calculated, where Pᵢ(c):c ε C_{T} is the probability of the pixel c belonging to class i. Table I lists the notation used in this description. FIG. 6 displays a flowchart of a methodology that includes the following three modules:

Module 1: Segmentation of the prostate on MRI is done prior to TRUS acquisition via a minimally interactive algorithm. In FIG. 6, the prostrate segmentation is shaded in pink.

Module 2: Create a multi-attribute probabilistic map of prostate location on TRUS. As an initial step attenuation correction is performed on the TRUS imagery. The probabilistic map is created by, (a) determining a spatial-based probability of the prostate on TRUS, (b) calculating a texture-based probability of the prostate on TRUS, and finally (c) estimating the probability of each pixel belonging to the prostate by combining the spatial and texture-based probabilities. In FIG. 6, blue corresponds to pixels least likely to belong to the prostate, red corresponds to pixels most likely to belong to the prostate.

Module 3: Register MRI prostate segmentation and TRUS probabilistic map. Registration is performed via an (a) affine transform to account for translation, rotation, and scale differences between images followed by (b) elastic transform to account for differences in prostate deformation.

The details of each Module will now be described in greater details:

### Module 1: Prostate Segmentation on MRI

Referring now to FIG. 7, the image processor 40 processes the MRI data to semi-automatically segment the prostate image data. The MRI segmentation includes the following steps.

1. Select subset of MRI data: An operator uses an input device, such as a mouse, stylus or touch screen to manually select a subset of image data that includes the prostate. For instance, the operator may use the input device to create a bounding box of the region containing the prostate.
2. Calculate Segmentation: The processor analyzes the selected subset of data to calculate the segmentation of the prostate within the bounding box region, using shape and appearance features.
3. Refine Segmentation: The segmentation may then be refined by selecting landmark points on the surface of the prostate. Specifically, the operator may analyze the MRI image and use an input device to select various landmark points. The landmark points constrain the processor to include these points on the surface of the prostate. In this way, the processor calculates the segmentation of the prostate in response to the selected landmark points.
4. Iterative Refinement: Steps 2 and 3 are repeated until an accurate segmentation is achieved. The accuracy of the MFA segmentation scheme is dependent on the selection of the bounding box (detailed in Step 1) and the landmark points (detailed in Step 3). In this way, a sensitivity analysis of the MFA prostate segmentation scheme is performed.

### Module 2: Probabilistic Model of Prostate Location on TRUS

As an initial step attenuation correction is performed on c_{T} image data to account for spatial variations in image intensity. A probabilistic map of prostate location on TRUS defined as Pᵢ(c) is then calculated by (1) extraction of texture features from c_{T} defined as F_{T}(c), and (2) estimation of the likely prostate location, referred to as the spatial prior and estimation of the likely prostate appearance, referred to as the texture prior.

Attenuation Correction

Attenuation correction is performed as follows. For each pixel c ε C_{T} with 3D Cartesian coordinates expressed as (x, y, z), defined such that the probe center is defined as x=0, y=0, z=0, a set of corresponding polar coordinates is calculated as follows:

$${r}^{2} = {x}^{2} + {y}^{2} \\ θ = {tan}^{- 1} \frac{x}{y} \\ z = z \qquad ( 8 )$$

Image attenuation is modeled within the polar coordinated reference frame as:

f_{T}(r.θ,z)=β(r,θ,z)\tilde{f}_{T}(r,θ,z)+η(r,θ,z) (9)

where \tilde{f}_{T}(r, θ, z) is the true, unknown TRUS signal associated with the location (r, θ, z). η(r, θ, z) is modeled as additive white Gaussian noise which is assumed to be independent of \tilde{f}_{T}(r, θ, z). Additionally, β(r, θ, z) may be estimated via convolution of a smoothing Gaussian kernel with the image, i.e. a low-pass filtering of the signal. The true underlying signal may then be recovered using the equation,

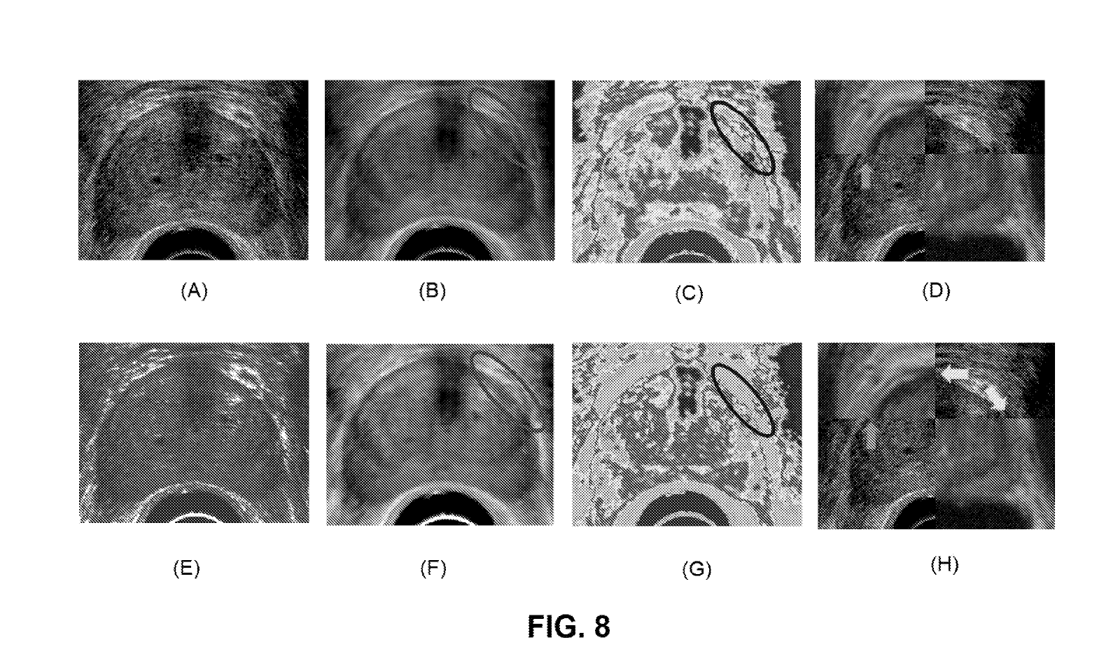

\tilde{f}_{T}(r,θ,z)=exp{log [f_{T}(r.θ,z)]−lpf(log [f_{T}(r.θ,z)]) (10)

where lpf is a low-pass filter. \tilde{f}_{T}(r, θ, z) is then converted back into 3D Cartesian coordinates, \tilde{f}_{T}(c): c ε C_{T}. FIG. 8(A)-8(H) illustrates results in which attenuation improved the results by over 1 mm.

Feature Extraction

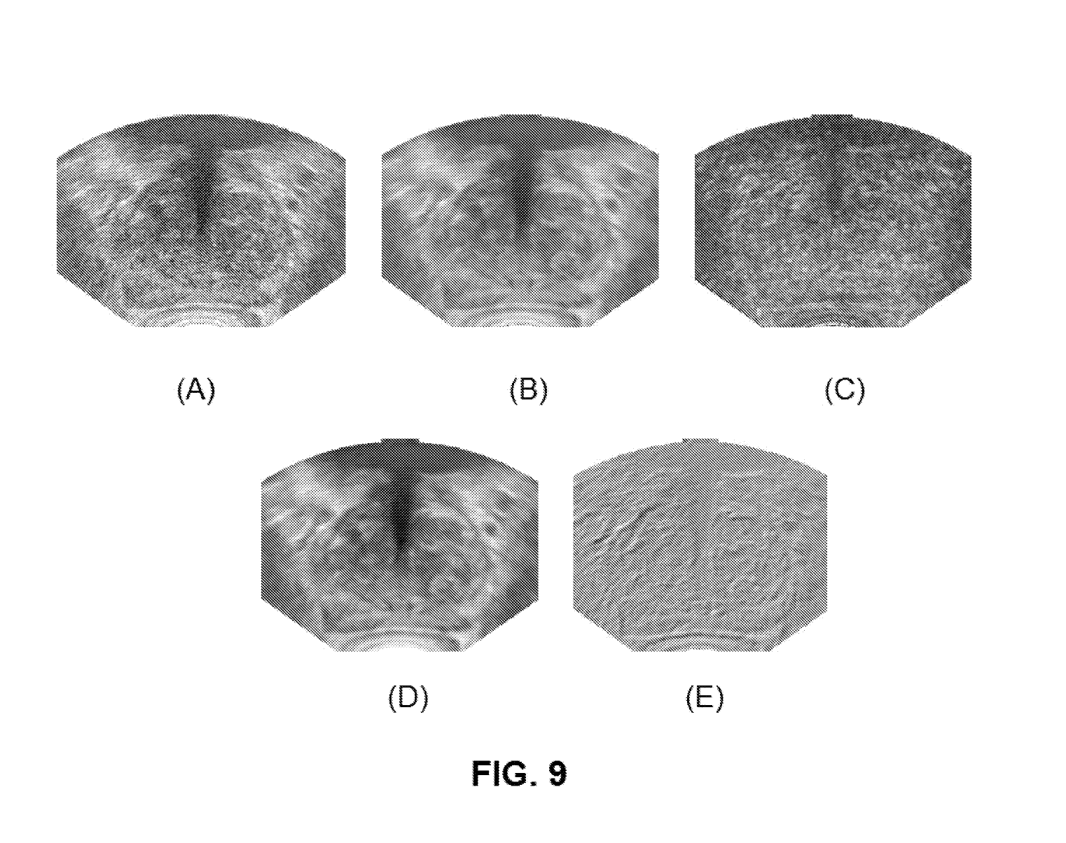

For each pixel \tilde{f}_{T}(c): c ε C_{T} a set of texture features F_{T}(c) are calculated. The texture features chosen describe (a) intensity for a pixel or a region (intensity, mean, median), (b) intensity spread in a region (range), (c) intensity variation (variance, Rayleigh, or the Nakagami m-parameter), (d) edge information (Gabor wavelet). FIGS. 9(A)-9(E) illustrate four representative texture features: (b) median, (c) range, (d) Rayleigh, and (e) Gabor wavelet.

Features that describe the intensity characteristics of a region are determined by defining a neighborhood of pixels N(c)f orc ε c_{T} and then calculating a texture feature value. For instance the mean intensity value is calculated as

$$fm ( c ) = \frac{1}{\vert N ( c ) \vert} {\sum }_{d ∈ N ( c )} \vert {\tilde{f}}_{T} ( d ) .$$

The median intensity value defined as f_{d}(c) is similarly calculated for the median filter operator. The range texture feature defined as fᵣ (c) describes the range of intensity values within N(c) for c ε C_{T}. The range texture feature value is calculated as

fᵣ(c)=max_{dεN(c)}(\tilde{f}_{T}(d))−min_{dεN(c)}(\tilde{f}_{T}(d)).

Intensity variation texture features are calculated to describe the spread of pixel intensity values assuming a specific underlying distribution. For instance the variance texture feature assumes that the underlying pixel distribution is Gaussian and is calculated as,

$${f}_{v} ( c ) = \sqrt{\frac{1}{\vert N ( c ) \vert} {\sum }_{d ∈ N ( c )} {( {\tilde{f}}_{T} ( d ) - {f}_{m} ( c ) )}^{2}} \qquad ( 11 )$$

Similarly, the Rayleigh texture feature assumes an underlying distribution that describes well formed ultrasound scatter and is defined as,

$${f}_{y} ( c ) = \sqrt{\frac{1}{2 \vert N ( c ) \vert} {\sum }_{d ∈ N ( c )} {{\tilde{f}}_{T} ( d )}^{2}} \qquad ( 12 )$$

The Nakagami m-parameter defined as fₙ describes the shape of a distribution that is generalizable across different scatter conditions on ultrasound. To calculate the Nakagami m-parameter an iterative method may be employed.

Finally, edge information is calculated using a set of texture features extracted from Gabor wavelets. Gabor wavelets are calculated by modulating a complex sinusoid with a Gaussian function. The Gabor wavelets when convolved with the TRUS imagery return high values for regions with strong edges and low values for regions with weak edges. The feature set F_{T}(c) is then set as a subset of [fₘ, f_{d}, fᵣ, fᵥ, f_{y}, fₙ, f_{g}].

Calculating Probability Map of Prostate Location on TRUS

The probability of pixel c belong to class i, defined as Pᵢ(c), is dependent on the location of c and the feature set F_{T}(c). The probability of a location c belonging to tissue class i is defined as pᵢ(c). Similarly the probability of a set of features F_{T}(c) belonging to tissue class i is pᵢ[F_{T}(c)]. pᵢ(c) and pᵢ[F_{T}(c)] are assumed to be independent so that the final probability Pᵢ(c) may be expressed as

Pᵢ(c)=pᵢ[F_{T}(c)]×pᵢ(c) (13)

The calculations of pᵢ(c) and pᵢ[F_{T}(c)] are described further below.

Spatial Probability:

pᵢ(c) is the likelihood of pixel c belonging to class i based on its spatial location. pᵢ(c) is calculated from a set of J training studies C_{T,j} : j ε {1, . . . , J}, where for each study the prostate has been delineated by an expert, yielding the 3D prostate segmentation M_{T,j}. The prostate segmentation is defined M_{T,j}=(C_{T},g_{T,j}) such that g_{T,j}(c)=i for a pixel c belonging to class i. The origin for each study is set as the center of the TRUS probe, so that the location of pixel c has a consistent position relative to the probe across all studies. pᵢ(c) which is the frequency of pixel c being located in the prostate across J training studies is defined as:

$${p}_{i} ( c ) = \frac{1}{J} {\sum }_{j = 0}^{J} {g}_{T , j} ( c ) \qquad ( 14 )$$

### Feature Probability:

The probability pᵢ[F_{T}(c)] is the likelihood of a set of features F_{T}(c) associated with pixel c, belonging to class i. In the present instance, F_{T}(c) is assumed to be accurately modeled as a multivariate Gaussian distribution with a mean vector of μ_{F,i} and a covariance matrix Σ_{F,i} for the ith class. Given the Gaussian distribution parameters μ_{F,i} and Σ_{F,i} the probability pᵢ[F_{T}(c)] is calculated as:

$${P}_{i} [ {F}_{T} ( c ) ] = \frac{1}{2 {π}^{k / 2} {\sum }_{F , i}^{1 / 2}} {e}^{{( {F}_{T} ( c ) - {μ}_{F , i} )}^{′} {\sum }_{F , i}^{- 1} ( {F}_{T} ( c ) - {μ}_{F , i } )} \qquad ( 15 )$$

Where k is the number of features in F_{T}(c). However, μ_{F,i} and Σ_{F,i} are unknown. Therefore, in the present instance, these two parameter are estimated.

To estimate μ_{F,i} and Σ_{F,i} the location of the prostate on TRUS is estimated. To estimate the location of the prostate on TRUS an initial rigid transformation Tᵣ is assumed. Using this, an estimated prostate segmentation is obtained, defined as \hat{M}_{T}=Tᵣ(M_{M}) where \hat{M}_{T}=(CT,ĝT) and ĝT (c)=i for a pixel c estimated to belong in class i. μ_{F,i} and Σ_{F,i} are then calculated as,

$${μ}_{F , i} = \frac{1}{\vert {Ω}_{T , i} \vert} {\sum }_{d ∈ {Ω}_{T , i}} {F}_{T} ( d ) ,$$

where Ω_{T,i} is the collection of pixels in C_{T} belonging to class i according to ĝT (c). Additionally, Σ_{F,i} is similarly defined for a covariance matrix of FT (d) for Ω_{T,i}.

### Module 3: Registration of MRI Segmentation and TRUS Probabilistic Model

A transformation T is identified to spatially map c_{M} onto c_{T}. In the present instance, T is calculated to align M_{M} and c_{T}. T is calculated via the equation:

T=argmax_{T}[S[T(M_{M}),c_{T}]−∝R(T)] (16)

where:
S[T(M_{M}),c_{T}] is a similarity metric between T(M_{M}) and c_{T}
R(T) is a regularization function that penalizes T for not being smoothly varying and
∝ reflects the weight of R relative to S(,.,).

The similarity metric S(,.,) is calculated as:

S[T(M_{M}),c_{T}]=Πᵢ₌₀¹Π_{cεC}_{_{T}}[pᵢ[F(c),c]|T(M_{M})=Ω_{M,i}] (17)

where: Ω_{M,i} is the collection of pixels in C_{M} belonging to class i.

T is initialized with a rigid transformation Tᵣ such that overlap between MM and P₁(c) is maximized. The rigid transformation is calculated as:

Tᵣ=argmax_{Tr}[Π_{cεC}_{_{T}}[P₁(c)×Tᵣ(g_{M}(c))]] (18)

Given the initial alignment Tᵣ, and affine registration Tₐ followed by an elastic registration Tₑ is used to align MRI and TRUS images.

Affine Registration

For the affine transformation Tₐ no regularization R(T) is used since Tₐ is by definition smoothly varying. Not defining R(T) is equivalent to setting ∝=0.

### Elastic Registration

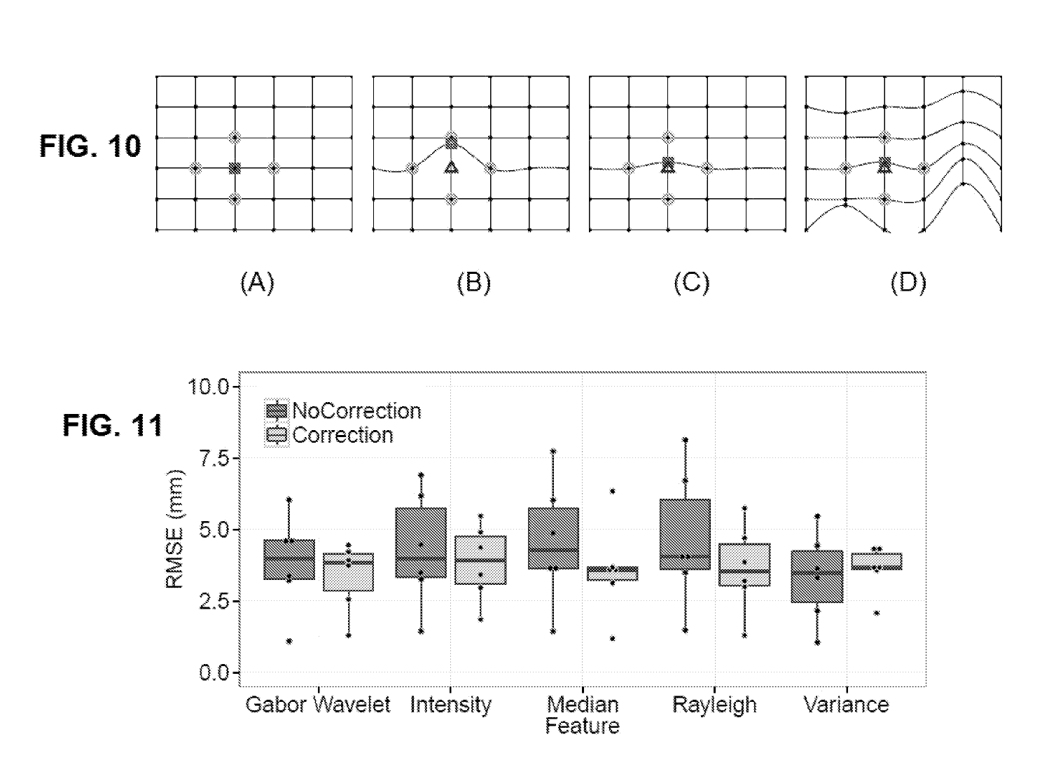

An elastic B-spline-based transformation Tₑ may be used to recover differences in prostate deformation between MRI and TRUS. Tₑ is defined by a set of knots which determine the Transformation Tₑ for all c ε C_{M}. Each knot, defined by its location p ε C_{M}, is allowed to move independently as shown in FIG. 10.

The term R(T) is added to constrain Tₑ to only those transformations that are likely to occur. R(T) is calculated as:

R(T)=Σ_{pεT}(1−e^{-∥p-E[p]∥}) (19)

where:
p is the location of a B-Spline knot and E[p] is the maximum likelihood estimate of where p should be located.

In the present instance, E[p] is estimated as:

$$E [ p ] = \frac{1}{\vert N ( p ) \vert} {\sum }_{q ∈ N ( p )} q \qquad ( 20 )$$

where:

N(p) is the set of knots which neighbor p.

Thus E[p] is the average over the set of knots which neighbor the knot at location p. FIG. 10 gives a 2D illustration of a regularization used in the present methodology. In the present instance, the regularization step is performed in 3D.

R(T) is defined such that if p=E[p], then the knot p will not contribute to the value of R(T). As p moves farther from E[p], the value of (1−e^{-∥p-E[p]∥) increases, and contributes more to the value of R(T). Hence R(T) is lower for evenly spaced, smoothly varying knots compared to randomly spaced, erratically varying knots. Deformations that are not evenly spaced and smoothly varying will only occur if they improve the similarity metric S(•,•).}

### EXPERIMENTAL DESIGN AND RESULTS

The MAPPER process described above was used on two different cohorts of MRI and TRUS. The first cohort comprised six patients with pelvic phase-array coil MRI and 2D ultrasound. The second cohort comprised seven patients with endorectal coil MRI and 3D ultrasound. For all studies a radiologist manually selected corresponding fiducials on the MRI and TRUS images. Corresponding fiducials included, the urethra, the center for those locations deemed suspicious for prostate cancer, and the center of small calcifications. In addition, a radiologist manually delineated the prostate boundary on MRI and TRUS.

1. Dataset 1 (D₁): Side-Firing Transrectal Probe

T2-weighted MRI was acquired using a Siemens 1.5 T scanner and a pelvic phased-array coil for 6 patients. TRUS imagery was acquired using a B-K Profocus probe that acquires 2D transverse B-mode images of the prostate. The TRUS probe was attached to a mechanical stepping device used to translate the probe perpendicular to the axial plane at 2 mm intervals. For each patient one TRUS volume was acquired, where each volume consists of a set of parallel B-mode slices. A single radiologist selected corresponding landmark points between all six MRI-TRUS pairs.

2. Dataset 2 (D₂): Volumetric End-Firing Transrectal Probe

T2-weighted MRI was acquired using a General Electric (GE) 3.0 T scanner and an endorectal coil for seven patients. TRUS imagery was acquired using a GE 4DE7C probe, that acquired 3D data in a single, multi-plane sweep of the prostate. For each patient 1-3 volumes were acquired, where each volume is acquired directly from the ultrasound device. A total of 13 MRI-TRUS pairs were acquired for the seven patients. Two radiologist selected corresponding landmark points between the MRI-TRUS pairs. The first radiologist selected corresponding landmark points for 10 studies and the second radiologist selected corresponding landmarks points for 5 studies.

Performance Evaluation: Root Mean Squared Error (RMSE)

RMSE is a measure of how well two corresponding point sets align; a RMSE of 0 represents perfect alignment. A manually selected set of fiducials on MRI were defined as p_{M}ⁱ : i ε {1, . . . , N}. Similarly, a set of fiducials on TRUS were defined as p_{T}ⁱ : i ε {1, . . . , N}, such that p_{M}ⁱ corresponds to p_{T}ⁱ. RMSE is then calculated as

$$\frac{1}{N} {\sum }_{i = 1}^{N} {( {p}_{M}^{i} - {p}_{T}^{i} )}^{2}$$

### Implementation Details

The methods described above were implemented using an Insight Segmentation and Registration Toolkit (ITK) version 4.5. Texture features were calculated using a N(c) with a spherical neighborhood of size 1 mm³, determined empirically to be large enough to accurately represent local image statistics while small enough to capture onlylocal image statistics. Both Ta and Te were found via a Powell optimization scheme using a single resolution.

### Results

### Experiment 1: Effect of Attenuation Correction on Registration Accuracy

It is possible that subtle differences in intensity characteristics across the TRUS image can lead to a probabilistic model Pᵢ(c) that does not accurately model the prostate location. Incorrect estimation of Pᵢ(c) can therefore result in sub-optimal image registration. The effect of attenuation correction was evaluated on registration accuracy in terms of RMSE. The MAPPER process was evaluated with and without attenuation correction for D₁.

FIG. 11 illustrates the quantitative results for Te, with and without attenuation correction for five texture features. Attenuation correction has two effects on the registration results: (1) attenuation reduces RMSE variance across studies and therefore gives a more robust image registration; and (2) attenuation lowers RMSE and, hence, provides a more accurate registration accuracy. The positive effects of attenuation correction on registration occur independent of which feature was used.

### Experiment 2: Selection of Regularization Weight

The regularization weight ∝ controls the relative importance of a smooth Tₑ and accurately registering the prostate mask on MRI to the TRUS probabilistic model (i.e. maximizing Equation 10). To assess the sensitivity of the performance of the MAPPER process on the choice of ∝, the regularization weight ∝ was varied for {100, 1, 1 E-2, 1 E-4} and assessed RMSE for D₁.

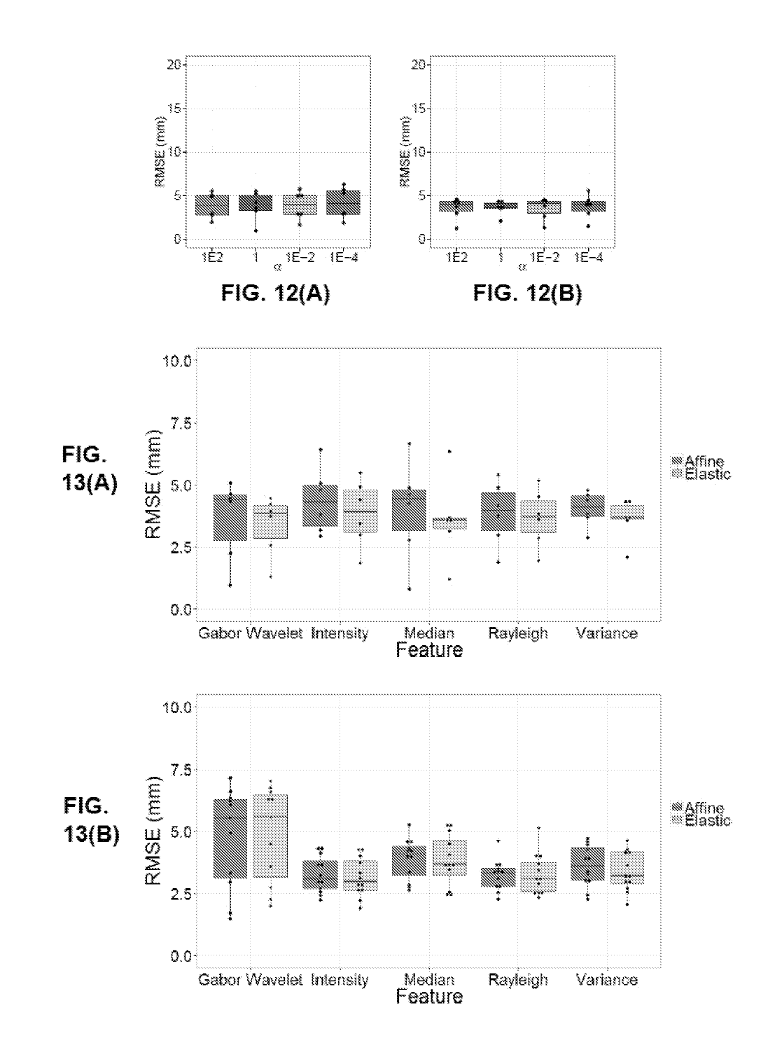

FIG. 12 illustrates the RMSE for each set of regularization parameters ∝ evaluated for two texture features, (a) Rayleigh and (b) variance. RMSE changes little with respect to ∝, even across a wide range of values, ∝ E {100, 1, 1E-2, 1E-4}.

### Experiment 3: Selection of Features for Creating Probabilistic Map of Prostate on TRUS

The accuracy of the probabilistic model Pᵢ(c) depends on the choice of features in F_{T}(c); those features which are best able to distinguish prostate from non-prostate tissue lead to a more accurate Pᵢ(c) and therefore to a more accurate image registration. In the foregoing description seven features are described in terms of RMSE for both datasets. Additionally, for D₂ we compared RMSE between expert radiologists to evaluate inter-observer variability.

FIG. 13 illustrates the RMSE for 5 of the 7 texture features evaluated for (a) D₁ and (b) D₂. Each dataset results in a different set of best performing features. For D₁, the side-firing TRUS probe, variance and Gabor wavelet texture features were best able to align the MRI and TRUS imagery. For D₂, the end-firing TRUS probe, the intensity and Rayleigh texture features were identified as the best features. The selection of different features from D₁ and D₂ most likely reflect differences in imaging characteristics between D₁ and D₂. Although the framework of the MAPPER process was able to align images with an average RMSE of approximately 3 mm, the results here reflects the positive effect of feature selection on accurate registration of the MRI and TRUS image data.

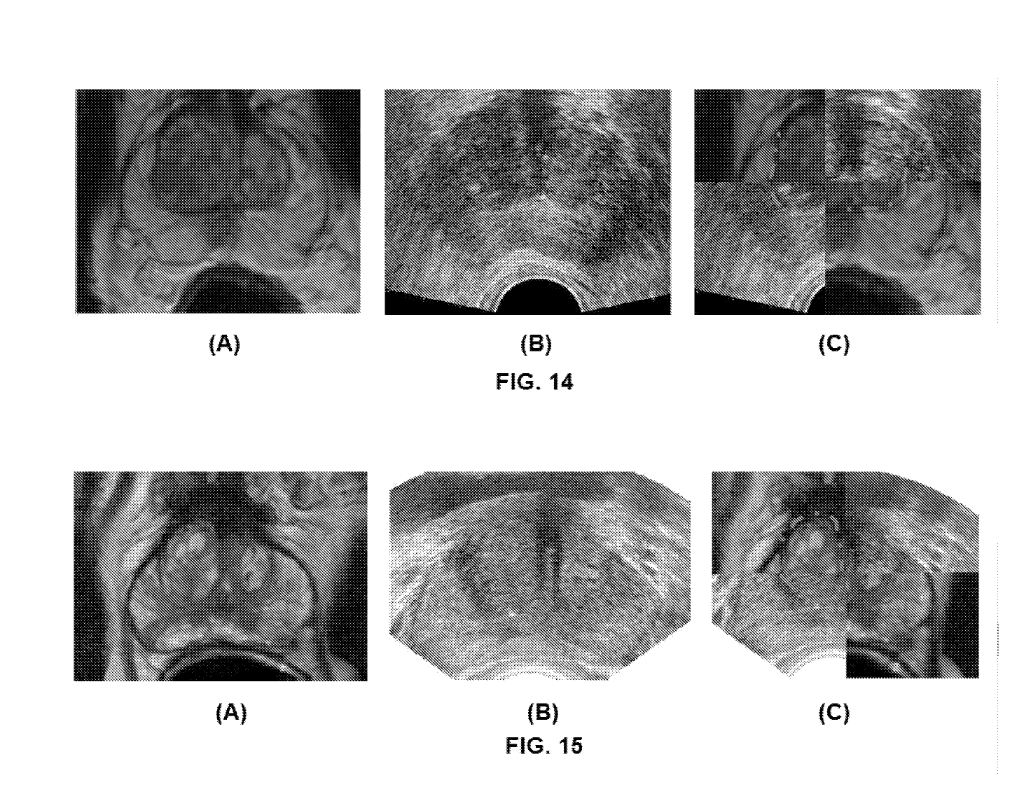

D₁, where MRI was acquired with a pelvic phase array coil, demonstrates an improvement in RMSE between Tₐ and Tₑ. In comparison D₂, where MRI data was acquired with an endorectal coil, had limited improvement in RMSE between Tₐ and Tₑ. These differences in RMSE improvement between Tₐ and Tₑ are indicative of D₁ having larger differences in prostate deformation between MRI and TRUS compared to D₂. FIG. 14 shows the registration result for a representative study from D₁ while FIG. 15 shows a corresponding registration result for a study from D₂. For both cases, the MAPPER process aligns the prostate surface as well as internal structures which are highlighted by dotted lines.

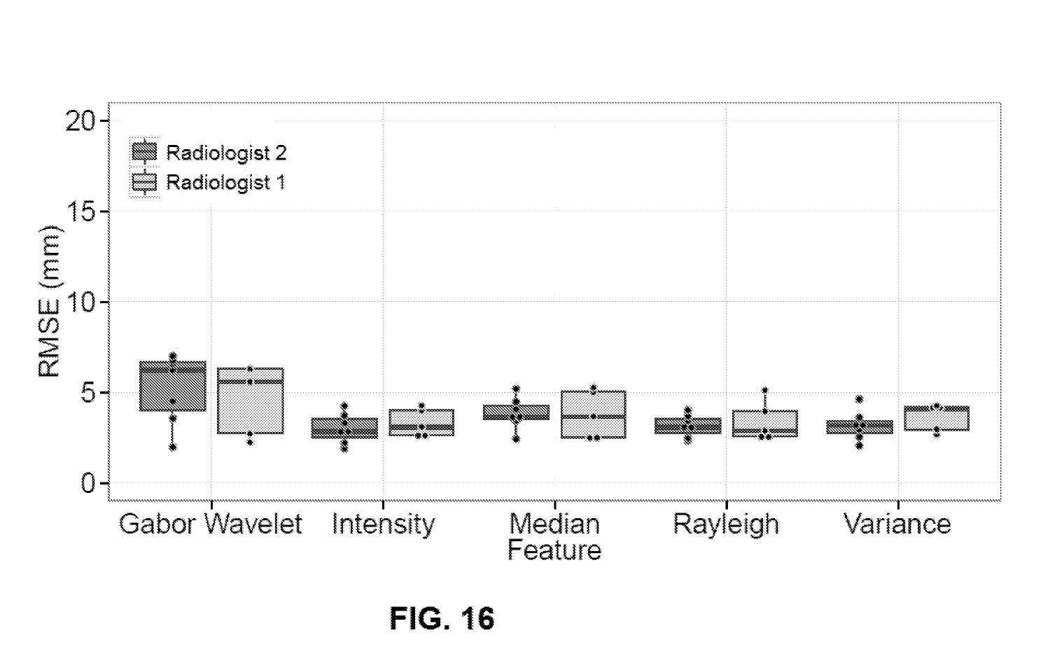

The MAPPER process on D₂ was also evaluated with respect to landmarks selected both on MRI and TRUS by two radiologists. FIG. 16 presents RMSE for each of two radiologists for Tₑ. For the best performing features (Rayleigh, intensity) the difference in RMSE between the two radiologists was roughly 0.3 mm.

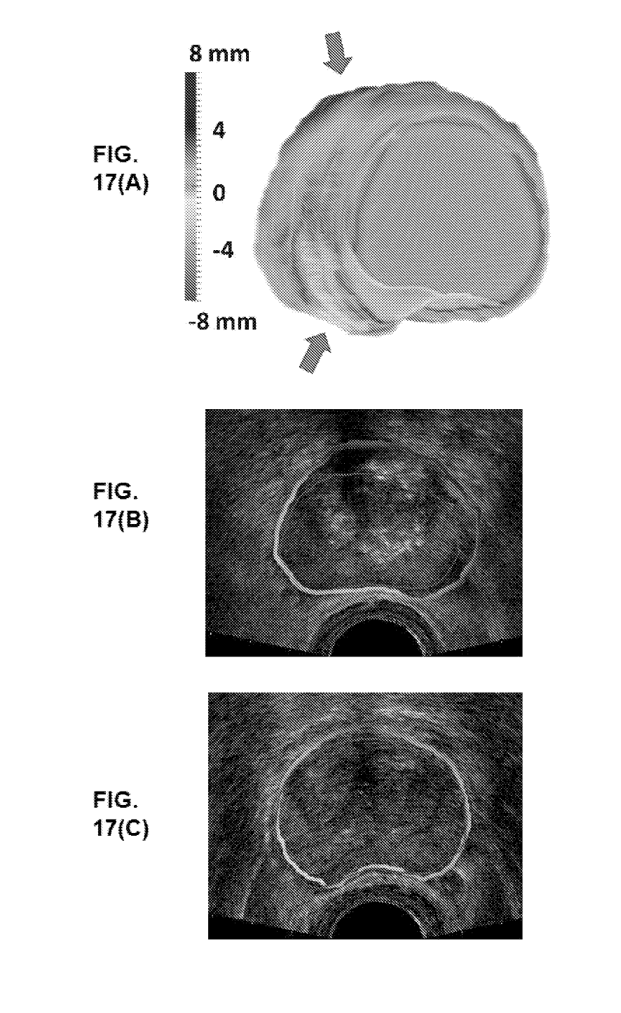

In the datasets analyzed, the MAPPER process showed an average RMSE of approximately 3 mm for D₁ and D₂. To further evaluate the accuracy of the MAPPER process surface renderings of the prostate surface were created, as shown in FIG. 17(A), where the blue and red represents regions where the MRI was misaligned external and internal to the prostate surface on TRUS respectively. In the example shown in FIG. 17(A) there are two regions of misalignment, near the rectal wall (yellow) and near the bladder (blue). FIG. 17(B) illustrates an axial plane of the TRUS, displayed with two boundaries overlaid. These represent the axial cross section of the surface rendering shown in FIG. 17(B)-(C) and the true prostate boundary (brown line).

The hyper-echoic region distal to the TRUS probe was caused due to the probabilistic model Pᵢ(c) not appropriately modeling the location of the prostate, resulting in a registration error of 4 mm. Similarly FIG. 17(C) illustrates a different axial plane of the TRUS, displayed with the cross section of the surface rendering shown in FIG. 17(A) and the true prostate boundary (brown line). This misalignment is much less pronounced, representing a registration error of ≈1 mm near the rectal wall the error is primarily because Tₑ did not fully account for the subtle differences in prostate deformation. As can be seen, poor TRUS image quality can negatively impact the registration results due to the fact that the MAPPER process is based in part on TRUS image appearance for the texture-based probability.

Experiment 4: Effects of Prostate MRI Segmentation Accuracy

The accuracy of MAPPER process was evaluated in the context of variation in segmentation performance on account of different levels of manual intervention to segment the prostate. For both D₁ and D₂ the two top performing texture features identified in Experiment 3 were used to perform this evaluation. Different levels of user interaction were evaluated via the following strategies.

Bounding box: Manual selection of bounding box of the region containing the prostate prior to MFA segmentation.

Manual correction: Manual selection of bounding box of the region containing the prostate and selection of landmark points to correct the automated segmentation if necessary.

Manual delineation: Manual delineation of the prostate on MRI by a radiologist.

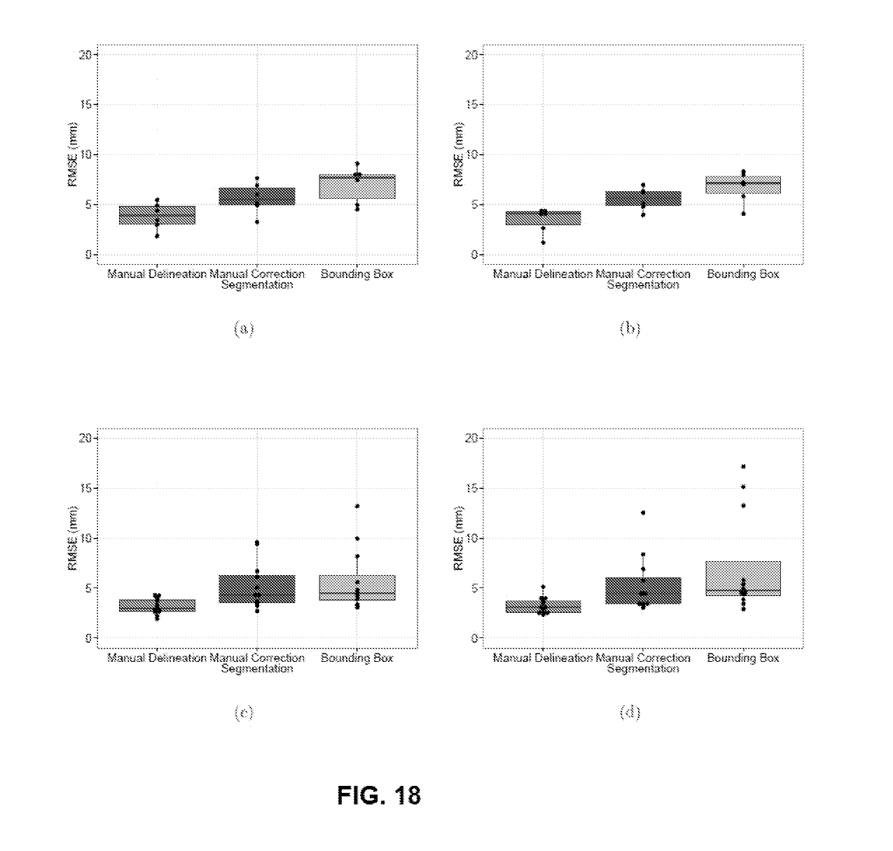

FIGS. 18(A)-18(D) illustrate registration accuracy, in terms of RMSE for each segmentation scheme. Manual prostate delineation, the most accurate segmentation scheme, also has the best registration accuracy. The manual correction of the semi-automated segmentation scheme resulted in an improved registration compared to the semi-automated scheme without manual intervention.

For D₂ there were outliers in terms of RMSE when utilizing a bounding box-based segmentation method.

MAPPER uses a semi-automated segmentation scheme on MRI in conjunction with a probabilistic map of the prostate location on TRUS to register MRI onto TRUS. Hence, the MAPPER process automatically detects and aligns the prostate on MRI and TRUS rather than relying on manual intervention to either delineate the prostate or select corresponding fiducials on MRI and TRUS.

The process as described above uses the B-Spline transformations in Module 3, which recover non-linear deformations with few additional constraints, to account for the difference in deformation of the prostate between MRI and TRUS imagery. In the present instance an additional regularization constraint was imposed to smoothly vary the underlying deformation in the prostate. However, other transformations such as Finite Element Models (FEM), which allow for explicit modeling of tissue physics, could also potentially be used to drive the MRI-TRUS fusion.

As evidenced by the results in Experiment 4, accurate segmentation of the prostate on MRI improves the accuracy of the MAPPER process. The prostate segmentation algorithm is performed offline prior to the biopsy procedure using a Multi-Feature Appearance (MFA) model of prostate appearance on MRI.

It will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

1. A method for registering image data between two images, comprising the steps of:
scanning a patient using a first imaging modality to obtain a first set of image data for a portion of the patient;
scanning a patient using a second imaging modality to obtain a second set of image data for the portion of the patient;
identifying a portion of the first set of image data corresponding to a portion of interest; and
analyzing the second set of data relative to the identified portion to identify a portion of the second image data corresponding to the portion of interest.

2. The method of claim 1 comprising the step of illustrating the portion of interest on a display of the second image data in response to the step of analyzing the second set of image data.

3. A method for registering image data of a patient from two imaging devices to guide a surgical procedure, wherein a first set of image data of the patient is provided by a first imaging device using a first imaging modality, wherein the method comprises the steps of:
scanning a patient using a second imaging device to obtain a second set of image data for a portion of the patient, wherein the second imaging device uses a second imaging modality that is different than the first imaging modality;
automatically processing the second set of image data to correlate the second set of image data with the first image data modality to align image data of a target tissue of the patient from the second imaging modality with image data of the portion of tissue from the first imaging modality; and
displaying the aligned image data from the first and second image data;
wherein the step of displaying is performed during the step of scanning.

4. The method of claim 3 wherein the step of scanning comprises scanning the patient using an ultrasound probe.

5. The method of claim 3 wherein the step of automatically processing the second set of image data comprises processing the image data by calculating the likelihood that a set of features associated with a data point of the second set belong to a subset of image data representing the target tissue.

6. The method of claim 5 wherein the set of features comprise one or more texture features.

7. The method of claim 6 wherein the texture features comprise one or more of: intensity for a pixel or a region, intensity spread in a region, intensity variation, edge information.

8. The method of claim 7 wherein the texture features comprise one or more of: mean, median, range, variance, Gabor wavelet, Rayleigh and Nakagami m-parameter.

9. The method of claim 6 wherein the step of processing the second set of image data comprises calculating the likelihood that the data point belongs to the subset of image data based upon the spatial location of the point relative to the second imaging device.

10. The method of claim 6 wherein the step of processing the second set of image data comprises correcting the data to account for attenuation caused by signal loss during the step of scanning.

11. The method of claim 3 wherein the step of processing the second set of image data comprises using an affine transformation to account for translation, rotation and scale differences between the image data from the first modality and the image data from the second modality.

12. The method of claim 11 wherein the step of processing the second set of image data comprises using an elastic transformation to account for differences in deformation of the target tissue between when the first image data was obtained from the first modality and when the second image data was obtained during the step of scanning.

13. A surgical apparatus, comprising:
a scanning element using a first imaging modality to scan a patient to obtain a first set of image data from a patient;
a cutting element for obtaining a tissue sample from the patient;
an image processor configured to process the first set of image data and correlate the first set of image data with a second set of image data from the patient obtained using a second imaging modality, wherein the image processor is configured to process the first set of image data to align image data of a target tissue of the patient from the scanning element with image data of the portion of tissue from the second set of image data; and
a video display for displaying the aligned image data from the first and second image data.

14. The apparatus of claim 13 wherein the image processor processes the first set of image data in real time to display the aligned image data during the step of scanning.

15. The apparatus of claim 13 wherein the scanning element comprises an ultrasound probe.

16. The apparatus of claim 15 wherein the cutting element comprises a biopsy needle or a laser ablation probe.

17. The apparatus of claim 13 wherein the image processor is configured to process the first set of image data by calculating the likelihood that a set of features associated with a data point of the first set of image data belong to a subset of image data representing the target tissue.

18. The apparatus of claim 17 wherein the set of features comprise one or more texture features.

19. The apparatus of claim 18 wherein the texture features comprise one or more of: mean, median, range, variance, Gabor wavelet, Rayleigh and Nakagami m-parameter.

20. The apparatus of claim 18 wherein the image processor is configured to calculate the likelihood that the data point belongs to the subset of image data based upon the spatial location of the point relative to the second imaging device.

21. The apparatus of claim 13 wherein the image processor is configured to correct the first image data to account for attenuation caused by signal loss during the step of scanning.

22. The apparatus of claim 13 wherein the image processor is operable to use an affine transformation to account for translation, rotation and scale differences between the image data from the first modality and the image data from the second modality.
